(19) United States
(12) Patent Application Publication
(10) Pub. No.: US 2026/0069729 A1
(43) Pub. Date: Mar. 12, 2026

(54) SELF DISINFECTING COUNTER

(71) Applicant: Max Enenstein, Los Angeles, CA (US)

(72) Inventor: Max Enenstein, Los Angeles, CA (US)

(21) Appl. No.: 18/884,038

(22) Filed: Sep. 12, 2024

(51) Int. Cl. A61L 2/10; A47B 33/00; A47F 10/06; A61L 2/24; A61L 2/26

(52) U.S. Cl. CPC A61L 2/10; A47B 33/00; A47F 10/06; A61L 2/24; A61L 2/26; A47B 2220/0077; A61L 2202/11; A61L 2202/121; A61L 2202/14

(57) ABSTRACT

The present system provides a counter that incorporates an integrated UV light for periodic use in disinfecting the counter. The counter has a clear top, comprised of a material that is transparent to UV light. The counter includes sensors that detect the presence of people near the counter. The sensors disable the UV light to prevent exposure of people to the light when in use. The sensors also prevent the UV light from turning on until there are no people present. The UV light in one embodiment has a timed cycle sufficient to disinfect the counter surface, making it usable for food service.

### BACKGROUND OF THE SYSTEM

Germs and contamination on food counters are a significant concern in food safety. Food counters, especially in kitchens and food preparation areas, as well as food pick up stations in stadiums and restaurants, can harbor a variety of harmful microorganisms, including bacteria like Salmonella, E. coli, and Listeria, as well as viruses and fungi. These germs can be introduced through raw foods, unwashed hands, dirty utensils, or contaminated surfaces. Once present, they can multiply rapidly, particularly in warm, moist environments.

Contamination can occur in several ways:

Cross-Contamination: This happens when harmful bacteria or other pathogens are transferred from one surface or food to another. For example, using the same cutting board for raw meat and vegetables without proper cleaning in between can spread bacteria.

Direct Contamination: Direct contact with contaminated surfaces, hands, or equipment can introduce germs to food. Even seemingly clean surfaces can be contaminated with invisible pathogens.

Airborne Contamination: Germs can spread through the air, landing on food counters and potentially contaminating food that is being prepared.

To prevent contamination, it's essential to regularly clean and disinfect food counters, use separate cutting boards for different types of food, and maintain good hygiene practices, such as washing hands frequently. Keeping food counters dry and properly storing food can also reduce the risk of germ growth. Proper food safety protocols are crucial to minimize the risk of foodborne illnesses.

Regular wiping and washing can help keep surfaces clean, but often introduce dangerous or unwanted chemicals onto food preparation and serving surfaces. In some cases, UV light can be used to help disinfect a surface.

UV light, particularly ultraviolet C (UVC) light, is widely used to decontaminate surfaces by destroying harmful microorganisms such as bacteria, viruses, and fungi. UVC light has a short wavelength (typically between 200-280 nanometers) and is highly effective at disrupting the DNA and RNA of these microorganisms, rendering them unable to replicate and causing them to die.

The use of UVC light for surface decontamination offers several advantages:

Effectiveness: UVC light can kill a broad spectrum of pathogens, including those resistant to other forms of disinfection. It can reach and disinfect surfaces that may be difficult to clean with traditional methods.

Non-Chemical: Unlike chemical disinfectants, UVC light does not leave behind residues, making it a cleaner option for sensitive environments such as food preparation areas, healthcare settings, and laboratories.

Time-Efficient: UVC decontamination can be done relatively quickly, making it suitable for high-traffic areas or situations where rapid turnaround is necessary.

However, there are also challenges and limitations to the use of UVC light:

Safety: Direct exposure to UVC light can be harmful to human skin and eyes, necessitating precautions to prevent accidental exposure.

Surface Shadows: UVC light only disinfects surfaces it directly contacts. Shadows or crevices where the light doesn't reach can remain contaminated.

Material Sensitivity: Prolonged exposure to UVC light can degrade certain materials, including plastics and fabrics, limiting its use in some applications.

Overall, UVC light is a powerful tool for surface decontamination, especially when used in conjunction with other cleaning methods, ensuring thorough and effective disinfection.

### SUMMARY

The present system provides a counter that incorporates an integrated UV light for periodic use in disinfecting the counter. The counter has a clear top, comprised of a material that is transparent to UV light. The counter includes sensors that detect the presence of people near the counter. The sensors disable the UV light to prevent exposure of people to the light when in use. The sensors also prevent the UV light from turning on until there are no people present. The UV light in one embodiment has a timed cycle sufficient to disinfect the counter surface, making it usable for food service.

### BRIEF DESCRIPTION OF THE DRAWINGS

FIG. 1 illustrates a counter in a fixed location.

FIG. 2 illustrates a counter on a food truck.

FIG. 3 illustrates an embodiment of the system with fixed UV lights.

FIG. 4 illustrates an embodiment of the system with moving UV lights.

FIG. 5 is a block diagram of an embodiment of the system.

FIG. 6 is a flow diagram illustrating the operation of the system in an embodiment.

### DETAILED DESCRIPTION OF THE SYSTEM

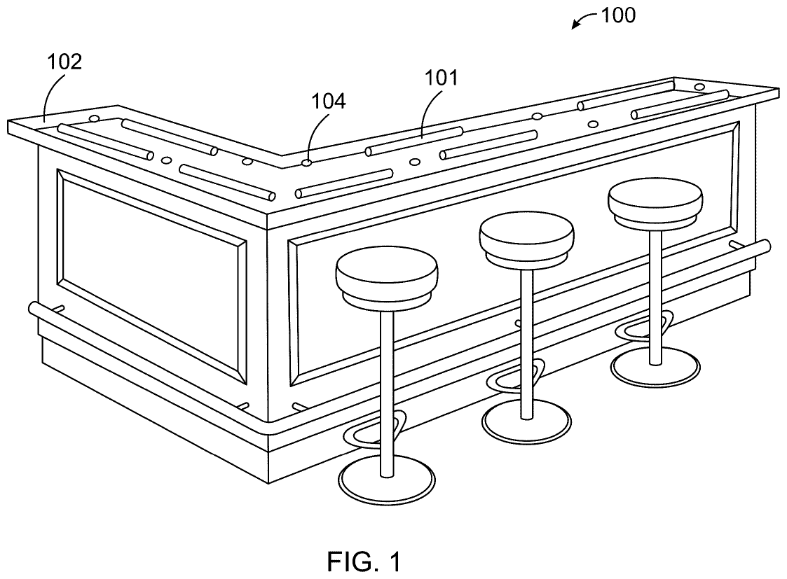

The system provides built in UV lights installed beneath a counter that is transparent to UV light. FIG. 1 illustrates the system incorporated in a food counter in a fixed location. A food service bar 100 is provided with a surface 102 that is transparent to UV light. In one embodiment, the system utilizes specific types of glass that are transparent to UV light especially in the UVC (100-280 nm) range. Such glass can include:

Fused Silica (Quartz Glass): Fused silica, also known as quartz glass, is highly transparent to UV light, including the UVC range. It is commonly used in UV lamps, optical lenses, and other applications requiring UV transmission.

UV-Grade Fused Silica: This is a purified form of fused silica specifically designed for maximum UV transparency. It offers excellent transmission across a wide UV spectrum, including deep UV and vacuum UV (VUV) ranges.

Borosilicate Glass: Borosilicate glass, such as Pyrex, is somewhat transparent to UVA and UVB light but has limited transmission in the UVC range. It is more transparent to UV light than regular soda-lime glass.

Specialty UV-Transmitting Glass: Some glass manufacturers produce specific types of glass designed to transmit UV light. These glasses are often used in scientific instruments, UV sterilization devices, and certain types of lighting.

Calcium Fluoride (CaF₂) and Magnesium Fluoride (MgF₂): While not technically “glass,” these crystalline materials are often used in optical components and are highly transparent to UV light, especially in the deep UV range.

Beneath the surface 102 are a plurality of UV lights 101. In one embodiment the UV lights are in the form of tubes and transmit UVC light. The UV lights 101 can be turned on to periodically and quickly disinfect the surface 102. The system also includes sensors 104 to prevent the UV light from being turned on when the sensors detect motion or otherwise detect the presence of a person within a desired range.

In operation, the UV lights are turned on for a desired amount of time to kill the germs, bacteria viruses, fungi, and the like so that a clean food surface is provided.

The amount of UVC exposure required to kill germs depends on several factors, including the type of microorganism, the intensity of the UVC light, and the duration of exposure. Generally, UVC doses are measured in microjoules per square centimeter (μJ/cm²), which combines the intensity of the light and the time of exposure.

Most bacteria, such as E. coli and Salmonella, typically require a UVC dose of around 2,000 to 10,000μJ/cm² for effective inactivation.

Viruses often require lower doses. For instance, the influenza virus can be inactivated with a dose of 2,000 to 5,000μJ/cm². More resilient viruses, like adenoviruses, may require higher doses, around 10,000 to 20,000μJ/cm².

Fungi and bacterial spores are generally more resistant and may require doses upward of 20,000 to 50,000μJ/cm².

Some factors influencing UVC include the effectiveness distance from the UVC Source: The effectiveness of UVC light decreases with distance, so closer proximity to the light source results in more effective disinfection.

Surface Characteristics: Smooth, non-porous surfaces are easier to disinfect with UVC light than rough, porous surfaces that may shield microorganisms in crevices.

Duration of Exposure: Longer exposure increases the dose of UVC light received by the microorganisms, making it more likely to effectively kill them.

A common UVC lamp emitting 253.7 nm light at an intensity of 1 mW/cm² can deliver a dose of 1,000μJ/cm² in just one second. To kill a typical bacterium requiring 10,000μJ/cm², it would take about 10 seconds of exposure. To kill most viruses would require 20 seconds and to kill fungi and spores would require 50 seconds or one minute.

In one embodiment, the food surface can be covered to prevent accidental exposure of people to the UV light. The cover is disinfected during each use, so does not contaminate the food counter surface during operation.

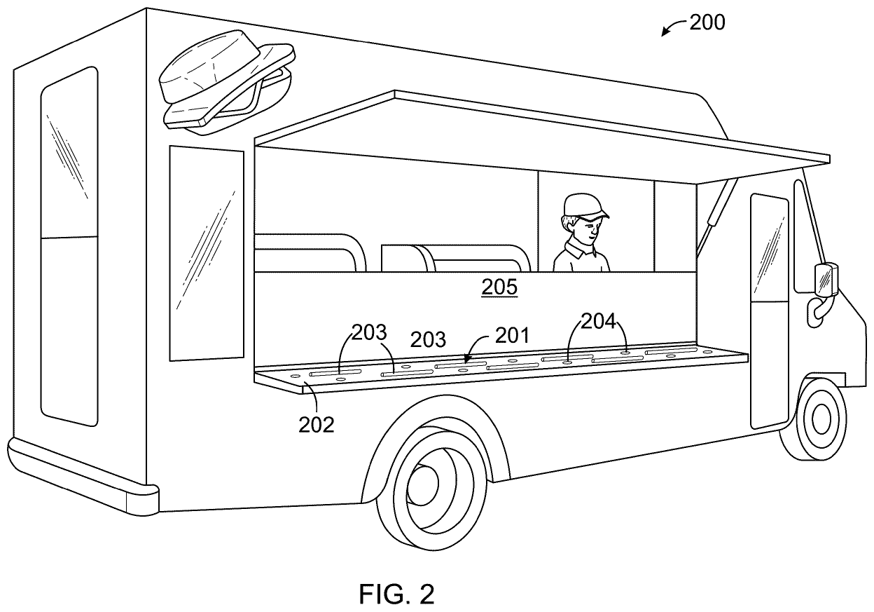

FIG. 2 illustrates a food truck having a food service area that incorporates the system. The truck 200 includes a food counter surface 201. The counter 201 includes a UVC transparent top surface 202, a plurality of UVC producing lights 203 and sensors 204. A hinged cover 205 can be lowered over the countertop during the cleaning cycle. The sensors detect when the top is lowered, and will not activate if the top is not lowered or if the sensors detect people nearby.

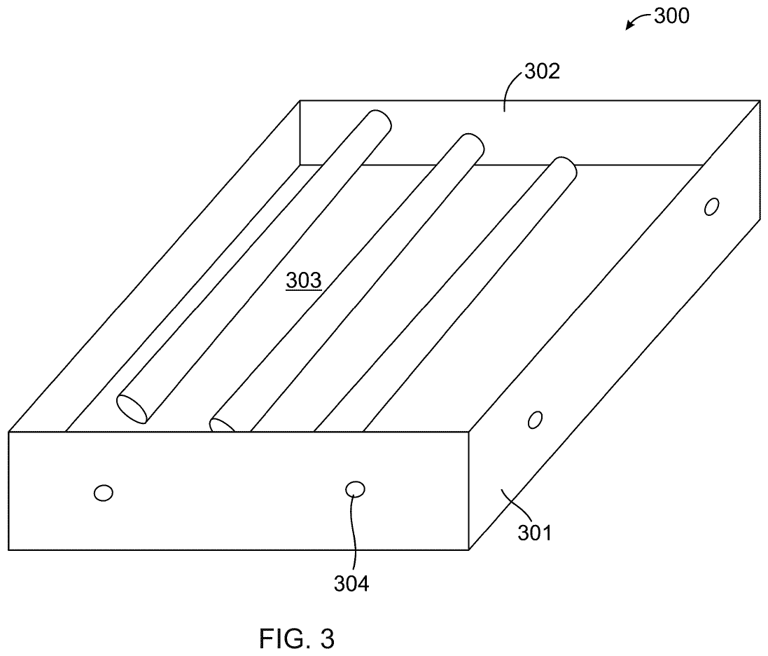

FIG. 3 is an exemplary embodiment of the self-cleaning counter 300. The counter comprises a box frame 301 that includes a UV transparent top 302. Within the frame a plurality of UV light sources 303 are disposed. Sensors 304 are disposed in multiple locations on the top and sides of the apparatus as needed to prevent activation of the UV light sources when people are present. In one embodiment, a UV opaque cover may be disposed over the surface of the counter during the UV disinfecting step.

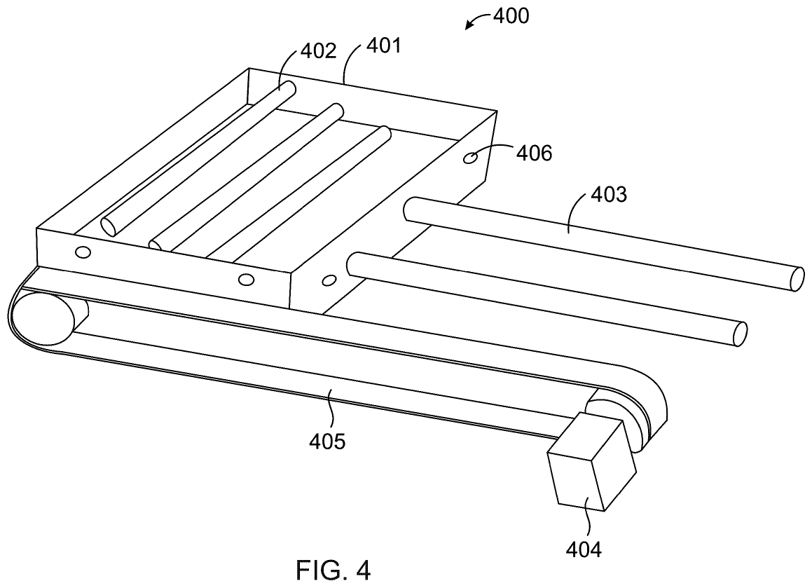

In one embodiment, the system uses stationary UV light sources. As shown in FIG. 4, the system may also be used with scanning type UV light sources. The scanner 400 includes a box 401 that includes one or more UV light sources 402. The box 401 is mounted on rails 403. A motor 404 rotates a belt 405 that pulls the box along the rails 403 to an end point and then reverses direction to send the box 401 back to an initial position. Sensors 406 serve to disable the UV light sources 402 when people are detected within a certain range.

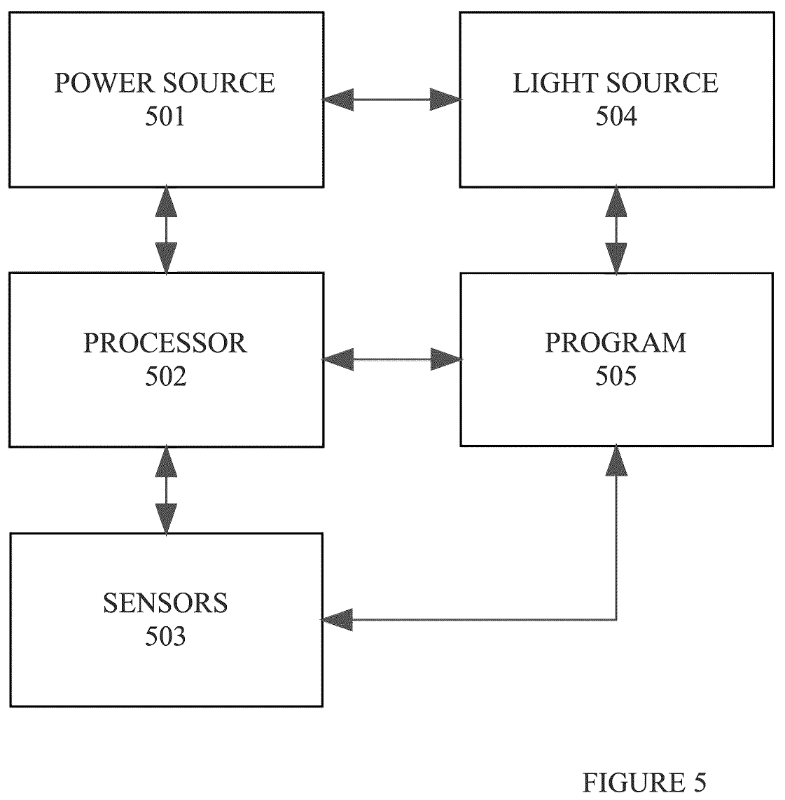

FIG. 5 illustrates an embodiment of the system. The system comprises a power source 501, processor 502, sensors 503, light source 504 and program 505. The processor 502 receives data from the program 505 and sensors 503. When power is provided, the processor 502 confirms that sensor data shows it is safe to turn on the light source 504. If so, the light source is enabled and the light source stays on pursuant to the program stored at 505. If the sensors do not provide the safe signal, the processor disables the light source 504.

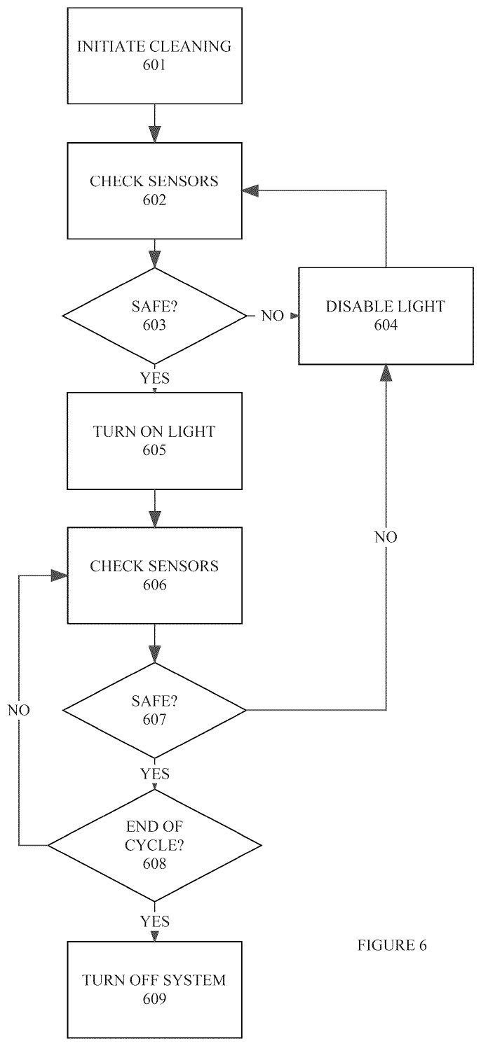

FIG. 6 is a flow diagram illustrating the operation of the system in an embodiment. At step 601 the user initiates a cleaning cycle. At step 602 the system checks the sensors to see if it is safe to clean (e.g. no risk of people nearby). At decision block 603 the system determines if the sensors are providing a safe signal. If not, the system proceeds to step 604 and disables the UV light, and returns to step 602.

If the sensors indicate safe at step 603, the system turns on the UV light at step 605. At step 606 the system again checks the sensors. At decision block 607 it is determined if the sensors indicate a safe environment. If not, the system goes to step 604, to disable the lights. If the sensors are safe at step 607, the system proceeds to decision block 608 to see if the cleaning cycle is over (e.g. the lights have been on for the desired amount of time). If not, the system returns to step 606. If the cycle is over at step 608, the system turns off at step 609.

What is claimed is:

1. An apparatus comprising:
a counter having a UV transparent surface;
a UV light source disposed within the counter and below the UV transparent surface;
a sensor for detecting the presence of a person within a predetermined range of the UV transparent surface;
a switch for turning on the UV light source to disinfect the UV transparent surface;
a processing means for turning off the UV light source when a person is detected within the predetermined range.

2. The apparatus of claim 1 wherein the UV light source provides UVC light.

3. The apparatus of claim 2 wherein the UV transparent surface is transparent to UVC light.

4. The apparatus of claim 1 further including cover for the UV transparent surface during the disinfecting of the UV transparent surface.

5. The apparatus of claim 1 further including programming means to determine a length of time for the UV light source to be on to disinfect the UV transparent surface.

6. The apparatus of claim 1 wherein the counter is a food preparation counter.

7. The apparatus of claim 1 wherein the counter is a food service counter.

8. The apparatus of claim 1 wherein the UV light source is stationary in the counter.

9. The apparatus of claim 1 wherein the UV light source moves within the counter.
